(19) United States
(12) Patent Application Publication
(10) Pub. No.: US 2025/0236640 A1
(43) Pub. Date: Jul. 24, 2025

(54) SEPARATION OF PRE-PEAK IN FUSION PROTEIN SAMPLE BY USING SIZE EXCLUSION HIGH PERFORMANCE LIQUID CHROMATOGRAPHY

(71) Applicant: KASHIV BIOSCIENCES, LLC, Piscataway, NJ (US)

(72) Inventors: Roshan Ganeshlal Upadhyay, Ahmedabad (IN); Darshana Jiten Maniar, Ahmedabad (IN); Shivani Singh, Ahmedabad (IN)

(21) Appl. No.: 18/699,511

(22) PCT Filed: Oct. 8, 2022
(86) PCT No.: PCT/IB2022/059648

§ 371 Date: Apr. 8, 2024

(30) Foreign Application Priority Data
Oct. 8, 2021 (IN) 202121045943

(51) Int. Cl. C07K 1/16; B01D 15/34; B01D 15/42; B01J 20/28; B01J 20/283; C07K 14/705

(52) U.S. Cl. CPC C07K 1/16; B01D 15/34; B01D 15/426; B01J 20/28083; B01J 20/283; C07K 14/70521; C07K 2319/30

(57) ABSTRACT

The present invention provides an effective High Performance Liquid Chromatography (SE-HPLC) method to separate or resolve the pre-peak and main peak (fusion protein). The method provides improved sharpness and resolution of pre-peak impurity. The method provides pre-peak area not less than 1.0 and resolution more than 1.3 in SE-HPLC. Moreover, the present invention also provides the method for the estimation and/or quantification of pre-peak and main peak of the protein mixture.

### FIELD OF THE INVENTION

The present invention provides an effective High Performance Liquid Chromatography (SE-HPLC) method to separate or resolve the pre-peak and main peak (fusion protein). The method provides improved sharpness and resolution of pre-peak impurity. The method provides pre-peak area not less than 1.0 and resolution more than 1.3 in SE-HPLC. Moreover, the present invention also provides the method for the estimation and/or quantification of pre-peak and main peak of the protein mixture.

### BACKGROUND OF THE INVENTION

In the production of biologics, it is very important to develop robust process to provide protein with high purity and less impurities especially high molecular weight impurities (HMWs). In order to establish a successful downstream process, it is very imperative to analyze the post-harvest protein mixture to evaluate or characterize the impurities such as HMWs. Size exclusion High Performance Liquid Chromatography is a technique to estimate or quantify pre-peak but resolving a pre-peak from main peak is very challenging and it is observed that routine Size exclusion High Performance Liquid Chromatography does not provide sharp resolution of pre-peak and main peak of complex proteins such as antibody or fusion proteins. In absence of obtaining a sharp resolution, it is very difficult for skilled person to quantify the presence of pre-peak adequately in the protein sample and it further creates uncertainty about the impurities during down-stream purification (DSP) which makes the DSP process expensive, ineffective, and lengthy. Therefore, the present invention is also directed to improve impurity resolution with at least more than 1.3 and improved impurity area more than 1. It is very important to develop an effective, robust Size exclusion High Performance Liquid Chromatography process to separate, estimate or quantify impurities such as HMW or pre-peak.

The present invention solves the problem and provide effective, robust Size exclusion High Performance Liquid Chromatography process to separate, estimate and/or quantify impurities present in fusion protein mixture such as high molecular weight impurities (HMWs).

### SUMMARY OF THE INVENTION

In an embodiment, the invention provides the process for performing Size exclusion High Performance Liquid Chromatography to estimate and/or quantify impurities such as HMWs present in protein mixture.

In an embodiment, the present invention provides a method for the separation of protein mixture comprising fusion protein of interest and pre-peak impurity, the process comprising;
a) loading the protein mixture onto Size exclusion High Performance Liquid Chromatography (SE-HPLC) column;
b) separating the protein mixture with suitable mobile phase comprising combination of salts at suitable pH higher than isoelectric point (pI) of the fusion protein; wherein the mobile phase maintains flow rate more than 0.3 mL/min and less than 0.6 mL/min.
c) separating the pre-peak from fusion protein; wherein the separation provides pre-peak area not less than 1.0 and resolution more than 1.3.

In an embodiment, the invention separates the pre-peak and main peak of fusion protein at suitable flow rate above 0.3 ml/min.

In an embodiment, the invention separates the pre-peak and main peak of fusion protein at suitable flow rate selected from 0.35 ml/min, 0.4 ml/min, about 0.45 ml/min, about 0.5 ml/min, about 0.55 ml/min, about 0.6 ml/min, about 0.65 ml/min about 0.7 ml/min, about 0.75 ml/min, and about 0.8 ml/min.

In an embodiment, the loading concentration of protein mixture is selected from about 0.5 mg/ml to about 1.4 mg/ml.

In an embodiment, the loading amount of protein mixture is selected from about 10 μg to about 100 μg.

In an embodiment, the protein mixture can be obtained selected from cell culture harvest, protein A eluate, mixed mode chromatography eluate, anion exchange chromatography eluate, cation exchange chromatography eluate or after any other purification steps.

In an embodiment, the protein mixture can be obtained from harvest, partially purified, substantially purified by any other purification methods.

In an embodiment, the protein mixture can be obtained from affinity chromatography, preferably protein A chromatography.

In certain embodiment, the suitable pH of mobile phase is selected from about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, and about 7.0.

In an embodiment, the SE-HPLC column comprising silica-based resin preferably diol type silica-based resin. In an embodiment, the column pore size is selected from 25 nm or 250 Å to 50 nm or 500 Å. In certain embodiment, the columns are selected from TSKgel G3000SWXL, TSKgel G4000SWXL, TSK gel UP-SW3000, BioSep-SEC-S2000, BioSep-SEC-S3000, BioSep-SEC-S4000.

In an embodiment, the column pore size is 25 nm or 250 Å.

In an embodiment, the invention provides USP peak tailing is from about 0.7 to about 1.15.

In certain embodiment, the pre-peak area is not merged or interfered with main peak area.

In an embodiment, the suitable detection absorbance is selected from about 214 nm to about 280 nm. In an embodiment, the detection absorbance is 215 nm.

In an embodiment, the invention provides purity of fusion protein of interest or main peak more than 98%.

In an embodiment, the salts are selected from sodium and potassium salt. In certain embodiment, the salts are selected from sodium sulphate, potassium chloride.

In certain embodiment, the mobile phase is free of sodium chloride, arginine, acetonitrile, TFA, guanidine hydrochloride, urea and formic acid.

In an embodiment, the present invention provides an improved method for quantification and/or estimation of impurities in a protein sample comprising;
a) a protein mixture comprising protein of interest and size variant impurities;
b) loading the protein mixture onto said Size exclusion High Performance Liquid Chromatography (SE-HPLC) column;
c) separating the protein mixture with suitable mobile phase comprising combination of salts at suitable pH 5.5 to 7.0; wherein the salt is selected from sodium and potassium salts;
d) analysed or quantified the pre-peak and main peak of the protein mixture at suitable detection absorbance.

In an embodiment, the present invention provides an improved method for quantification and/or estimation of impurities in a protein sample comprising;
a) a protein mixture from harvest comprising protein of interest and size variant impurities;
b) loading the protein mixture onto said Size exclusion High Performance Liquid Chromatography (SE-HPLC) column;
c) separating the protein mixture with suitable mobile phase comprising combination of salts at suitable pH 5.5 to 7.0; wherein the salts are potassium phosphate in combination with potassium chloride in suitable concentration;
d) analysed or quantified the pre-peak and main peak of the protein mixture; wherein the SE-HPLC column provides the resolution of pre-peak area not less than 1.0 and resolution more than 1.3.

In such embodiment, the concentration of mobile phase Potassium phosphate is selected from about 50 mM, about 60 mM, about 65 mM, about 70 mM, about 75 mM, about 80 mM, about 85 mM, about 90 mM, about 95 mM, about 100 mM, about 105 mM, about 110 mM, 115 mM and about 120 mM, about 125 mM, about 130 mM, about 135 mM, about 140 mM, about 145 mM, about 150 mM.

In such embodiment, the concentration of mobile phase Potassium chloride is selected from about 50 mM, about 60 mM, about 70 mM, about 80 mM, about 90 mM, about 100 mM, about 120 mM, about 130 mM, about 140 mM, about 150 mM, about 160 mM, about 170 mM, about 180 mM, about 190 mM, about 200 mM, about 220 mM, about 230 mM and about 240 mM, about 250 mM.

In an embodiment, the invention provides a method for the separation of protein mixture comprising fusion protein and pre-peak impurity, the process comprises;
a) loading the protein mixture onto Size exclusion High Performance Liquid Chromatography (SE-HPLC) column;
b) separating the protein mixture with suitable mobile phase comprising combination of salts at suitable pH higher than isoelectric point of the fusion protein selected from pH 5.5 to about pH7.0; wherein the salts are Sodium phosphate in combination with Sodium sulphate in suitable concentration;
c) separating the pre-peak from fusion protein; wherein the separation provides pre-peak area not less than 1.0 and resolution more than 1.3.

In an embodiment, the present invention provides an improved method for quantification and/or estimation of impurities in a protein sample comprising;
a) a protein mixture from harvest comprising protein of interest and size variants impurities;
b) loading the protein mixture onto said Size exclusion High Performance Liquid Chromatography (SE-HPLC) column;
c) separating the protein mixture with suitable mobile phase comprising combination of salts at suitable pH; wherein the salts are Sodium phosphate in combination with Sodium sulphate in suitable concentration;
d) analysed or quantified the pre-peak and main peak of the protein mixture; wherein the SE-HPLC column provides the resolution of pre-peak area not less than 1.0 and resolution more than 1.3.

In such embodiment, the concentration of mobile phase Sodium phosphate is selected from about 50 mM, about 60 mM, about 65 mM, about 70 mM, about 75 mM, about 80 mM, about 85 mM, about 90 mM, about 95 mM, about 100 mM, about 105 mM, about 110 mM, 115 mM and about 120 mM.

In such embodiment, the concentration of mobile phase Sodium sulphate is selected from about 50 mM, about 60 mM, about 65 mM, about 70 mM, about 75 mM, about 80 mM, about 85 mM, about 90 mM, about 95 mM, about 100 mM, about 105 mM, about 110 mM, 115 mM and about 120 mM.

In an embodiment, the present invention provides an improved method for quantification and/or estimation of impurities in a protein sample comprising;
a) a protein mixture from harvest comprising CTLA4-IgG1 and size variant impurities;
b) loading the protein mixture onto said Size exclusion High Performance Liquid Chromatography (SE-HPLC) TSK gel Gu00swxl column;
c) separating the protein mixture with suitable mobile phase comprising combination of 100 mM Potassium phosphate with 200 mM potassium chloride at pH 6.5;
d) analysed or quantified the pre-peak and main peak of the protein mixture; wherein the SE-HPLC column provides the resolution of pre-peak area not less than 1.0 and resolution more than 1.3.

In an embodiment, the present invention provides an improved method for quantification and/or estimation of impurities in a protein sample comprising;
a) a protein mixture from harvest comprising CTLA4-IgG1 and size variant impurities;
b) loading the protein mixture onto said Size exclusion High Performance Liquid Chromatography (SE-HPLC) column which is TSK gel G3000swxl column;
c) separating the protein mixture with suitable mobile phase comprising combination of 100 mM Sodium phosphate with 100 mM Sodium sulphate at pH 6.5;
d) analysed or quantified the pre-peak and main peak of the protein mixture; wherein the SE-HPLC column provides the resolution of pre-peak area not less than 1.0 and resolution more than 1.3.

### BRIEF DESCRIPTION OF FIGURES

FIG. 1 shows the comparative effect of two mobile phase 100 mM Sodium phosphate with 100 mM Na2SO4, pH 6.5 and 100 mM Potassium phosphate with 200 mM KCl, pH 6.5 in TSK gel G3000swxl column.

FIG. 2 shows the linear response of loading amount of the sample in terms of total area in the range of 20 μg to 80 μg injection amount.

FIG. 3 shows the linear response of loading amount of the sample in terms of pre-peak area in the range of 20 μg to 80 μg injection amount.

FIG. 4 shows the effect on the pre-peak and main peak of the Sample (Reference CTLA4-IgG1 fusion protein) when treated with reducing agent DTT.

FIG. 5 shows the effect on the pre-peak and main peak of the post-harvest sample (CTLA4-IgG1 fusion protein) when treated with reducing agent DTT.

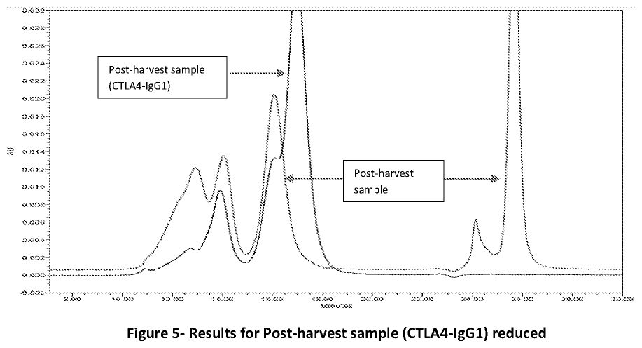

FIG. 6 shows the effect on the pre-peak and main peak of the Sample (Reference CTLA4-IgG1 fusion protein) when treated with IdeS.

FIG. 7 shows the effect on the pre-peak and main peak of the post-harvest sample (CTLA4-IgG1 fusion protein) when treated with IdeS.

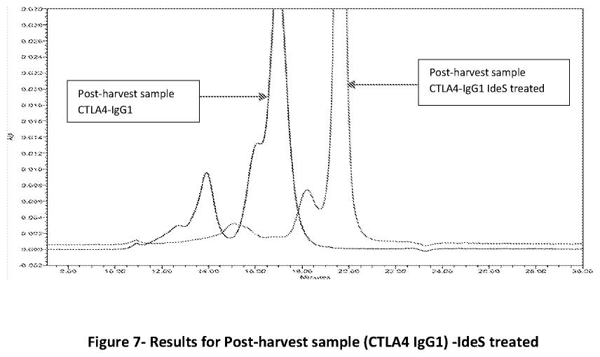

FIG. 8 shows the effect on the pre-peak and main peak of the Sample (Reference CTLA4-IgG1 fusion protein) when treated with PNGase F.

FIG. 9 shows the effect on the pre-peak and main peak of the post-harvest sample (CTLA4-IgG1 fusion protein) when treated with PNGase F.

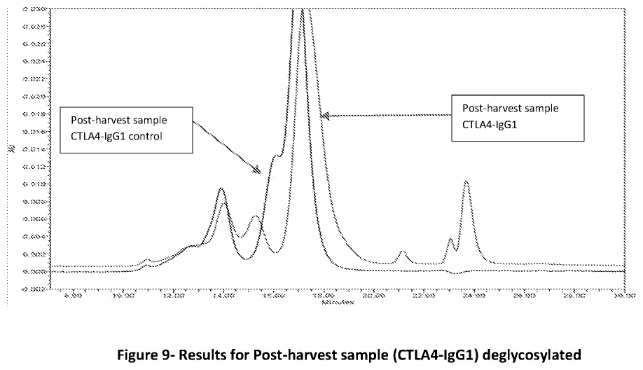

FIG. 10 shows the effect of flow rate at 0.5 mL/min. on the pre-peak area.

FIG. 11 shows the effect of flow rate at 0.3 mL/min. on the pre-peak area.

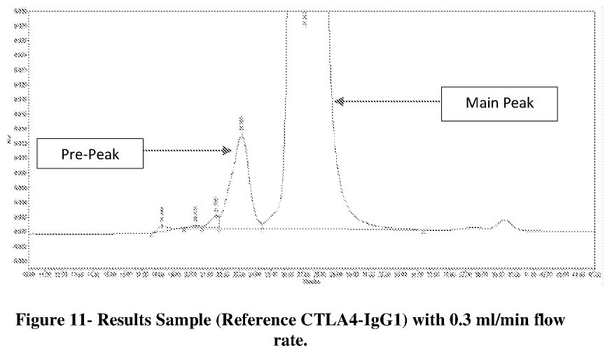

### DETAIL DESCRIPTION OF THE INVENTION

The present invention relates to an improved method for analysis of protein mixture comprises of at least one antibody or fusion protein, wherein the analysis of protein mixtures is performed with Size Exclusion High Performance Liquid Chromatography (SE-HPLC).

The term “Size Exclusion High Performance Liquid Chromatography” or “SE-HPLC” refers to chromatography processes that employs porous particles in the column to separate molecules by virtue of their size in solution. SE-HPLC is generally used to separate biological molecules, to determine molecular weight distributions of proteins. The chromatography column has silica-based resin preferably diol type silica-based resin.

In certain embodiment, the column pore size is more than 12.5 nm. In an embodiment, the column pore size is selected from about 25 nm or 250 Å to 50 nm or 500 Å.

In certain embodiment, the column pore size is more than 25 nm or 250 Å.

In certain embodiment, the columns are selected from TSKgel G3000SWXL, TSKgel G4000SWXL, TSK gel UP-SW3000, BioSep-SEC-S2000, BioSep-SEC-S3000, BioSep-SEC-S4000. In an embodiment, the TSKgel G3000SWXL is used for experimental purpose but any skilled person can use column similar chemistry to TSKgel G3000SWXL.

In certain embodiment, the size variants of the CTLA4-IgG1 fusion protein can be separated by SE HPLC and purity of the main peak of CTLA4-IgG1 fusion protein can be determined. The separation can be achieved by using size exclusion column with isocratic elution using a mobile phase and detection by UV at 215 nm.

The term “TSKgel G3000SWXL” used herein refers to a hydrophilic diol-type silica-based Size exclusion chromatography which has pore size 25 nm or 250 Å and dimension selected from 150*4.6 mm, 300*7.8 mm.

As used throughout the specification and in the amended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

The term “CTLA4-IgG1” or “CTLA4-IgG1 fusion protein” or “fusion protein of interest” or “fusion protein” used herein are interchangeable refers to a recombinant DNA generated fusion protein used to treat the symptoms of rheumatoid arthritis and to prevent joint damage caused by these conditions. CTLA4-IgG1 fusion protein is a biological product developed for immunosuppression by blocking T cell activation through inhibition of costimulatory signals and is indicated for treatment of rheumatoid arthritis. CTLA4-IgG1 fusion protein is a soluble homodimeric fusion protein of two identical subunits covalently linked by one disulfide bond. Each subunit consists of the modified amino acid sequence of the human cytotoxic lymphocyte associated antigen 4 (CTLA4), human immunoglobin IgG1 hinge, CH2 and CH3 region (Fc). Modification to the original sequences were introduced to avoid unintended disulfide bond formation and to reduce the ability of complement activation. Fusion protein examples such as TNF receptor 2-Fc (etanercept), rilonacept (Arcalyst—an IL-1 Trap), vascular endothelial growth factor trap (aflibercept), CTLA4-Fc fusion proteins (Abatacept and belatacept).

The term “protein mixture” and “protein sample” are interchangeable respectively in the present invention.

The term “Percentage (%) purity” refers to the percent of purity that determine the purity of protein present in the sample.

The term used “Percentage (%) purity” or “main peak area percentage (%)” and “main peak” are interchangeable respectively in the present invention refers to the CTLA4-IgG1 protein.

The term “Percentage (%) molecular weight related impurities” refers to percent of high molecular weight impurities.

The term “pre-peak area percentage (%)” refers to the percent of peak area that comes before the main peak area. The pre-peak area includes high molecular weight aggregates.

The term used “high molecular weight” or “HMW” or “HMWs” is product-related impurities that contribute to the size heterogeneity of fusion protein drug product. The formation of HMW species within a therapeutic fusion protein drug product as a result of protein aggregation can potentially compromise both drug efficacy and safety (e.g., eliciting unwanted immunogenic response). HMW is considered critical quality attribute that are routinely monitored during drug development and as part of release testing of purified drug product during manufacturing. In certain embodiment the HMW relates to aggregates.

The term “pI” or “Isoelectric point” used herein are interchangeable refers to the pH of a solution at which the net charge of a protein becomes zero. At solution pH that is above the pI, the surface of the protein is predominantly negatively charged, and therefore like-charged molecules will exhibit repulsive forces. Likewise, at a solution pH that is below the pI, the surface of the protein is predominantly positively charged, and repulsion between proteins occurs. The pI of CTLA4-IgG1 is less than 6.5.

The term “column” refers to the column of SE-HPLC selected from bioZen SEC-2, bioZen SEC-3, MabPac SEC-1, BioBasic SEC 60, BioBasic SEC 120, YMC SEC Mab, YMC-Pack Diol-200, TSK gel G3000swxl, and TSK gel G2000swxl.

The term “mobile phase” or “mobile phase buffer” are interchangeable refers to mobile phase having salts selected from sodium phosphate, sodium sulphate, potassium phosphate, potassium chloride, calcium chloride, and calcium phosphate.

The term “buffer” used herein refers to the solution comprising sodium phosphate, sodium sulphate, potassium phosphate, and potassium chloride.

The term “loading amount” refers to the amount of sample injected in the column during the process.

The term “flow rate” refers to amount of mobile phase passing through the column in unit time.

The term “solution stability” refers to stability of standard solution. Solution stability is determined by comparison for % purity and % molecular weight related impurities done for different timepoints. To evaluate solution stability, sample was diluted to 1 mg/ml in mobile phase and stored at 4-8° C. in HPLC autosampler.

The term “IdeS” refers to imlifidase, an endopeptidase which specifically and efficiently cleave IgG and results in fragments generation.

In an embodiment, the present invention comprises use of endoglycosidase which removes N-linked glycans. The endoglycosidase selected from Endoglycosidase F1, Endoglycosidases F2, Endoglycosidases H, Endo-α-N-acetylgalactosaminidase, and PNGase F.

In preferred embodiment, the endoglycosidase is PNGase F.

The term “PNGase F” refers to Peptide: N-glycosidase F, an endoglycosidase which specifically removes N-linked glycans. It allows the complete and rapid deglycosylation of antibodies and fusion proteins in only minutes.

The term “DTT” refers to Dithiothreitol, DTT is used to reduce the disulfide bonds of proteins and to prevent intramolecular and intermolecular disulfide bonds from forming between cysteine residues of proteins.

In an embodiment, the fusion protein is selected from CTLA4-IgG1, TNFR-IgG1, VEGF-IgG1. In certain embodiment, the isoelectric point (pI) of the fusion protein or fusion protein mixture is more than 5, 5.2, 5,4, 5.5, 5.6, 5.7, 5.8, 5.9, 6.0, 6.1, 6.2, 6.3, 6.4, 6.5, 6.6, 6.7, 6.8, 6.9, 7.0.

In another embodiment, the column used for SE-HPLC selected from MabPac SEC-1, YMC SEC Mab, from TSKgel G3000SWXL, TSKgel G4000SWXL, TSK gel UP-SW3000, BioSep-SEC-S2000, BioSep-SEC-S3000, BioSep-SEC-S4000.

In preferred embodiment, the column used for SE-HPLC is TSK gel G3000swxl.

In an embodiment, mobile phase having salts selected from sodium phosphate, sodium sulphate, potassium phosphate, and potassium chloride.

In other embodiment, the mobile phase having salts selected from sodium phosphate in combination with sodium sulphate, potassium phosphate in combination with potassium chloride, sodium phosphate in combination with potassium chloride, and potassium phosphate in combination with sodium sulphate.

In certain embodiment, the fusion protein sample is stable for 48 hours. In an embodiment the fusion protein sample is tested within 48 hours.

In an embodiment, the mobile phase is selected from sodium phosphate in combination with sodium sulphate, potassium phosphate in combination with potassium chloride, sodium phosphate in combination with potassium chloride, and potassium phosphate in combination with sodium sulphate in suitable concentration selected from about 50 mM to about 250 mM.

In an embodiment, mobile phase having salts are potassium phosphate in combination with potassium chloride.

In another embodiment, mobile phase having salts are sodium phosphate in combination with sodium sulphate.

In an embodiment, the salt concentration used in mobile phase is selected from about 50 mM, about 60 mM, about 65 mM, about 70 mM, about 75 mM, about 80 mM, about 85 mM, about 90 mM, about 95 mM, about 100 mM, about 105 mM, about 110 mM, 115 mM, about 120 mM, about 125 mM, about 130 mM, about 135 mM, about 140 mM, about 145 mM, and about 150 mM of Potassium phosphate.

In another embodiment, the salt concentration used in mobile phase is selected from about 80 mM, about 90 mM, about 100 mM, about 110 mM, and about 120 mM of Potassium phosphate.

In preferred embodiment, the salt concentration used in mobile phase is about 100 mM of Potassium phosphate.

In an embodiment, the salt concentration used in mobile phase is selected from about 100 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, and about 250 mM of Potassium chloride.

In another embodiment, the salt concentration used in mobile phase is selected from about 100 mM, about 150 mM, about 200 mM, and about 250 mM of Potassium chloride.

In preferred embodiment, the salt concentration used in mobile phase is about 200 mM of Potassium chloride.

In an embodiment, the salt concentration used in mobile phase is selected from about 50 mM, about 60 mM, about 70 mM, about 75 mM, about 80 mM, about 85 mM, about 90 mM, about 95 mM, about 100 mM, about 105 mM, about 110 mM, about 115 mM, about 120 mM, about 130 mM, about 140 mM, and about 150 mM of Sodium phosphate.

In another embodiment, the salt concentration used in mobile phase is selected from about 80 mM, about 90 mM, about 100 mM, about 110 mM and about 120 mM of Sodium phosphate.

In preferred embodiment, the salt concentration used in mobile phase is about 100 mM of Sodium phosphate.

In an embodiment, the salt concentration used in mobile phase is selected from about 100 mM, about 120 mM, about 130 mM, about 140 mM, about 150 mM, about 160 mM, about 170 mM, about 180 mM, about 190 mM, about 200 mM, about 210 mM, and about 220 mM of Sodium sulphate.

In another embodiment, the salt concentration used in mobile phase is selected from about 100 mM, about 150 mM, and about 200 mM of Sodium sulphate.

In preferred embodiment, the salt concentration used in mobile phase is about 200 mM of Sodium sulphate.

In an embodiment, the pH of mobile phase is adjusted to pH selected from about pH 5.5 to about pH 7.5, about pH 6.3 to about pH 7.5, about pH 6.5 to about pH 7.5, and about pH 6.7 to about pH 7.5.

In preferred embodiment, the pH of mobile phase is adjusted to about pH 6.5±0.05.

In an embodiment, the pH of mobile phase is adjusted to about pH 6.5±0.05 by acid selected from sulphuric acid, hydrochloric acid (HCl), nitric acid, and phosphoric acid.

In another embodiment, the pH of mobile is adjusted to about pH 6.5±0.05 by acid selected from hydrochloric acid (HCl) and phosphoric acid.

In preferred embodiment, the pH of mobile is adjusted to about pH 6.5±0.05 by Orthophosphoric acid.

In an embodiment, the flow rate of mobile phase is selected from about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min, and about 1.0 mL/min.

In another embodiment, the flow rate of mobile phase is selected from about 0.1 mL/min, about 0.2 mL/min, about 0.3 mL/min, about 0.4 mL/min, and about 0.5 mL/min.

In an embodiment, the flow rate of mobile phase is less than 0.6 mL/min.

In preferred embodiment, the flow rate of mobile phase is about 0.5+0.2 mL/min.

In an embodiment, the loading amount of sample injected in the column is selected from about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, and about 100 μg.

In another embodiment, the loading amount of sample injected in the column is selected from about 10 μg, about 20 μg, about 30 μg, about 40 μg, about 50 μg, about 60 μg, about 70 μg, about 80 μg, about 90 μg, and about 100 μg.

In preferred embodiment, the loading amount of sample injected in the column is selected from about 20 μg, about 30 μg, about 50 μg and about 80 μg.

In an embodiment, solution stability is determined at different time points selected from about 0 hr, about 1 hr, about 2 hrs, about 3 hrs, about 4 hrs, 5 hrs, about 6 hrs, about 7 hrs, about 8 hrs, about 9 hrs, about 10 hrs, about 11 hrs, about 12 hrs, about 13 hrs, about 14 hrs, about 15 hrs, about 16 hrs, about 17 hrs, about 18 hrs, about 19 hrs, about 20 hrs, about 21 hrs, about 22 hrs, about 23 hrs, about 24 hrs, about 25 hrs, about 26 hrs, about 27 hrs, about 28 hrs, about 29 hrs, about 30 hrs, about 31 hrs, about 32 hrs, about 33 hrs, about 34 hrs, about 35 hrs, about 36 hrs, about 37 hrs, about 38 hrs, about 39 hrs, about 40 hrs, about 41 hrs, about 42 hrs, about 43 hrs, about 44 hrs, about 45 hrs, about 46 hrs, about 47 hrs, about 48 hrs, about 49 hrs, about 50 hrs, about 51 hrs, about 52 hrs, about 53 hrs, about 54 hrs, about 55 hrs, about 56 hrs, about 57 hrs, about 58 hrs, about 59 hrs, and about 60 hrs.

In another embodiment, solution stability is determined at different time points selected from about 0 hr, about 6 hrs, about 12 hrs, about 18 hrs, about 24 hrs, about 30 hrs, about 36 hrs, about 42 hrs, about 48 hrs, about 52 hrs, and about 60 hrs.

In preferred embodiment, solution stability is determined at different timepoints about 0 hr, about 12 hrs, about 24 hrs, about 36 hrs, and about 48 hrs.

In an embodiment, the present invention comprises use of reducing agent that reduces the disulfide bonds of proteins. The reducing agent selected from TCEP-HCl, 2-Mercaptoethanol, Urea, and DTT.

In preferred embodiment, the reducing agent is DTT.

In an embodiment, the present invention comprises use of the endopeptidase which cleaves antibody and generates fragments. In an embodiment, the endopeptidase selected from Caspase-1, Papain, cathepsin K and IdeS.

In preferred embodiment, the endopeptidase is IdeS.

In an embodiment, the present invention provides a method for the separation of protein mixture comprising fusion protein of interest and pre-peak impurity, the process comprising;
a) loading the protein mixture onto Size exclusion High Performance Liquid Chromatography (SE-HPLC) column;
b) separating the protein mixture with suitable mobile phase comprising combination of salts at suitable pH higher than isoelectric point (pI) of the fusion protein; wherein the mobile phase maintains flow rate more than 0.3 mL/min and less than 0.6 mL/min.
c) separating the pre-peak from fusion protein; wherein the separation provides pre-peak area not less than 1.0 and resolution more than 1.3.

In an embodiment, the invention separates the pre-peak and main peak of fusion protein at suitable flow rate above 0.3 ml/min.

In an embodiment, the invention separates the pre-peak and main peak of fusion protein at suitable flow rate selected from 0.35 ml/min, about 0.3 ml/min about 0.4 ml/min, about 0.45 ml/min, about 0.5 ml/min, about 0.55 ml/min, about 0.6 ml/min, about 0.65 ml/min and about 0.7 ml/min.

In an embodiment, the loading concentration of protein mixture is selected from about 0.5 mg/ml to about 1.4 mg/ml.

In an embodiment, the loading concentration of protein mixture is selected from about 0.8 mg/ml to about 1.2 mg/ml.

In an embodiment, the loading concentration of protein mixture is 1.0 mg/ml.

In an embodiment, the loading amount of protein mixture is selected from about 10 μg to about 100 μg.

In an embodiment, the loading amount of protein mixture is selected from about 20 μg to about 80 μg.

In an embodiment, the loading amount of protein mixture is selected from about 40 μg to about 60 μg.

In an embodiment, the loading amount of protein mixture is about 30 μg.

In an embodiment, the protein mixture can be obtained selected from cell culture harvest, protein A eluate, mixed mode chromatography eluate, anion exchange chromatography eluate, cation exchange chromatography eluate or after any other purification steps.

In certain embodiment, the suitable pH of mobile phase is selected from about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0.

In certain embodiment, the suitable pH of mobile phase is 6.5.

In an embodiment, the present invention provides an improved method for quantification and/or estimation of impurities in a protein sample comprising;
a) a protein mixture from harvest comprising protein of interest and size variant impurities;
b) loading the protein mixture onto said Size exclusion High Performance Liquid Chromatography (SE-HPLC) column;
c) separating the protein mixture with suitable mobile phase comprising combination of salts at suitable pH 5.5 to 7.0;
d) analysed or quantified the pre-peak and main peak of the protein mixture at suitable detection absorbance.

In an embodiment, the present invention provides an improved method for quantification and/or estimation of impurities in a protein sample comprising;
a) a protein mixture comprising protein of interest and size variant impurities;
b) loading the protein mixture onto said Size exclusion High Performance Liquid Chromatography (SE-HPLC) column;
c) separating the protein mixture with suitable mobile phase comprising combination of salts at suitable pH 5.5 to 7.0; wherein the salt is selected from phosphate, sodium and potassium salts;
d) analysed or quantified the pre-peak and main peak of the protein mixture at suitable detection absorbance.

In certain embodiment, the pre-peak area is not merged or interfered with main peak area.

In an embodiment, the suitable detection absorbance is selected from about 214 nm to about 280 nm. In an embodiment, the detection absorbance is 215 nm.

In an embodiment, the SE HPLC column is TSKgel G3000SWXL.

In an embodiment, the present invention provides an improved method for quantification and/or estimation of impurities in a protein sample comprising;
a) a protein mixture from harvest comprising protein of interest and size variant impurities;
b) loading the protein mixture onto said Size exclusion High Performance Liquid Chromatography (SE-HPLC) column;
c) separating the protein mixture with suitable mobile phase comprising combination of salts at suitable pH; wherein the salts are Potassium phosphate in combination with potassium chloride in suitable concentration;
d) analysed or quantified the pre-peak and main peak of the protein mixture wherein the SE-HPLC column provides pre-peak area not less than 1.0 and resolution more than 1.3.

In such embodiment, the concentration of mobile phase Potassium phosphate is selected from about 60 mM, about 65 mM, about 70 mM, about 75 mM, about 80 mM, about 85 mM, about 90 mM, about 95 mM, about 100 mM, about 105 mM, about 110 mM, 115 mM, about 120 mM, about 125 mM, about 130 mM, about 135 mM, about 140 mM, about 145 mM, and about 150 mM.

In such embodiment, the concentration of mobile phase Potassium chloride is selected from about 100 mM, about 120 mM, about 130 mM, about 140 mM, about 150 mM, about 160 mM, about 170 mM, about 180 mM, about 190 mM, about 200 mM, about 220 mM, about 230 mM and about 240 mM, and about 250 mM.

In an embodiment, the present invention provides an improved method for quantification and/or estimation of impurities in a protein sample comprising;
a) a protein mixture from harvest comprising protein of interest and size variant impurities;
b) loading the protein mixture onto said Size exclusion High Performance Liquid Chromatography (SE-HPLC) column;
c) separating the protein mixture with suitable mobile phase comprising combination of salts at suitable pH; wherein the salts are Sodium phosphate in combination with Sodium sulphate in suitable concentration;
d) analysed or quantified the pre-peak and main peak of the protein mixture; wherein the SE-HPLC column provides pre-peak area not less than 1.0 and resolution more than 1.3.

In such embodiment, the concentration of mobile phase Sodium phosphate is selected from about 70 mM, about 75 mM, about 80 mM, about 85 mM, about 90 mM, about 95 mM, about 100 mM, about 105 mM, about 110 mM, 115 mM, and about 120 mM.

In such embodiment, the concentration of mobile phase Sodium sulphate is selected from about 70 mM, about 75 mM, about 80 mM, about 85 mM, about 90 mM, about 95 mM, about 100 mM, about 105 mM, about 110 mM, 115 mM, and about 120 mM.

In an embodiment, the present invention provides an improved method for quantification and/or estimation of impurities in a protein sample comprising;
a) a protein mixture from harvest comprising CTLA4-IgG1 and size variant impurities;
b) loading the protein mixture onto said Size exclusion High Performance Liquid Chromatography (SE-HPLC) column TSK gel G3000swxl column;
c) separating the protein mixture with suitable mobile phase comprising combination of 100 mM Potassium phosphate with 200 mM potassium chloride at pH 6.5;
d) analysed or quantified the pre-peak and main peak of the protein mixture; wherein the SE-HPLC column provides pre-peak area not less than 1.0 and resolution more than 1.3.

In an embodiment, the present invention provides an improved method for quantification and/or estimation of impurities in a protein sample comprising;
a) a protein mixture from harvest comprising CTLA4-IgG1 and size variant impurities;
b) loading the protein mixture onto said Size exclusion High Performance Liquid Chromatography (SE-HPLC) column which is TSK gel G3000swxl column;
c) separating the protein mixture with suitable mobile phase comprising combination of 100 mM Sodium phosphate with 100 mM Sodium sulphate at pH 6.5;
d) analysed or quantified the pre-peak and main peak of the protein mixture; wherein the SE-HPLC column provides pre-peak area not less than 1.0 and resolution more than 1.3.

In an embodiment, the pre-peak separates from about 12 minutes to about 20 minutes.

In an embodiment, the pre-peak separates within about 15 minutes.

The present invention provides an example for illustration purpose which should not be considered to limit the scope of the present invention with the described examples.

### EXAMPLES

Process for estimation and/or quantification of pre-peak and main peak of protein mixture comprising CTLA4-IgG1 fusion protein.

Reagents details:
a) Sodium phosphate dibasic anhydrous
b) Sodium phosphate monobasic monohydrate
c) Sodium sulphate anhydrous
d) Orthophosphoric acid
e) Potassium phosphate dibasic anhydrous
f) Potassium phosphate monobasic anhydrous
g) Potassium chloride
h) Milli Q water

Equipment details:
a) HPLC system equipped with a pump, an autosampler, a UV detector and a suitable data acquisition system
b) Digital Dry bath
c) Magnetic stirrer
d) pH meter
e) Analytical weighing balance
f) Sonicator
g) Filter assembly
h) 0.2 μm membrane filter

### EXAMPLE 1

Quantification of molecular weight related impurities and purity determination of protein mixture containing CTLA4-IgG1 fusion protein.

Sample (CTLA4-IgG1 fusion protein) was diluted from 25 mg/ml to 1 mg/ml in mobile phase. 30 μg sample was injected (injection volume 30 μl).

### Chromatographic Conditions:

| | |
|---|---|
| HPLC system: | HPLC system equipped with a pump, an autosampler, |
| | a UV detector and a suitable data acquisition system |
| Column: | TSK gel G3000swxl |
| Mobile Phase: | Mobile phase (with TSKgel G3000swxl): 100 mM |
| | potassium phosphate in combination with 200 mM |
| | KCl, pH 6.5 |
| Mode: | Isocratic |
| Detection: | UV at 215 nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

The experiment is performed by incorporating injection/s of the blank solution followed by injection/s of reference protein standard onto chromatographic column TSKgel G3000swxl. Test sample which is CTLA4-IgG1 is injected onto TSK G3000swxl thereafter.

TABLE 1
Results for Percentage (%) purity or Main peak area percentage (%)
and pre-peak area percentage (%) in TSK gel G3000swxl column:

| | Total Pre-peak | | USP | |
|---|---|---|---|---|
| | area percentage | Percentage | resolution | USP |
| Column | (%) | (%) purity | of pre-peak | tailing |
| TSK gel | 1.29 | 98.71 | 2.03 | 1.13 |
| G3000swxl | | | | |

As shown in Table 1, TSK gel G3000swxl column provides 98.71% purity, 1.29% of total pre-peak area of CTLA4-IgG1 fusion protein and resolution of pre-peak is 2.03.

### EXAMPLE 2

Comparison of mobile phase for quantification of pre-peak and main peak of protein mixture containing CTLA4-IgG1 fusion protein.

For SE HPLC, mobile phase with either sodium or potassium salt can be used. Sodium or potassium salts with different salt concentration were used with TSK column. Resolution of impurities was compared between different mobile phases.

Sample (CTLA4-IgG1 fusion protein) was diluted from 25 mg/ml to 1 mg/ml in mobile phase, 30 μg sample was injected (injection volume 30 μl).

### Chromatographic Conditions:

| | |
|---|---|
| HPLC system: | HPLC system equipped with a pump, an autosampler, |
| | a UV detector and a suitable data acquisition system |
| Column: | TSK gel G3000swxl |
| Mobile Phase: | Mobile phase with column TSK gel G3000swxl: |
| | a) 100 mM Sodium phosphate in combination with |
| | 100 mM Na2SO4, pH 6.5 |
| | b) 100 mM Potassium phosphate in combination with |
| | 200 mM KCl, pH 6.5 |
| Mode: | Isocratic |
| Detection: | UV at 215 nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

In present example, all the column conditions were kept constant except the mobile phase. Applicant has tried both the above-mentioned mobile phase a) and b) to observe the effect of mobile phase over the quantification of pre-peak and main peak of protein mixture containing CTLA4-IgG1 fusion protein.

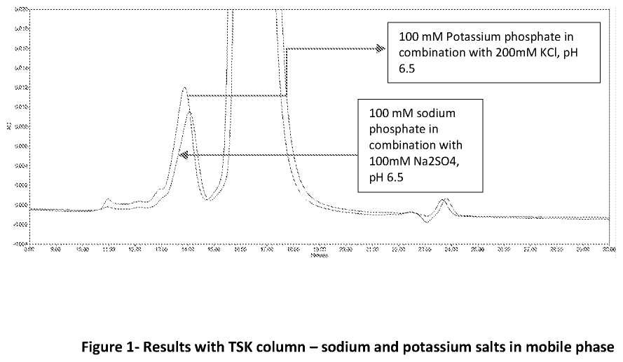

Based on the result shown in chromatographic profile (FIG. 1), it appeared that TSK gel G3000swxl column mobile phase with potassium salts i.e., 100 mM Potassium phosphate in combination with 200 mM KCl, pH 6.5, showed adequate pre-peak area or pre-peak area percentage (%), good purity and sharp resolution of pre-peak and main peak of protein mixture containing CTLA4-IgG1 fusion protein.

### EXAMPLE 3

Loading amount linearity for quantification of pre-peak and main peak of protein mixture containing CTLA4-IgG1 fusion protein.

In the initial method development experiments, 30 μg protein amount was injected to the column. In this parameter, CTLA4-IgG1 fusion protein was injected in the range of 20 μg to 80 μg to the column and linearity was evaluated in terms of total area and pre-peak area (HMWs area). Percentage (%) purity and total pre-peak area percentage (%) can be checked with different loading amount.

Sample (CTLA4-IgG1 fusion protein) was diluted from 25 mg/ml to 1 mg/ml in mobile phase. 20 μg, 30 μg, 50 μg and 80 μg sample was injected with injection volume of 20 μl, 30 μl, 50 μl, and 80 μl respectively.

### Chromatographic Conditions:

| | |
|---|---|
| HPLC system: | HPLC system equipped with a pump, an autosampler, |
| | a UV detector and a suitable data acquisition system |
| Column: | TSK gel G3000swxl |
| Mobile Phase: | Mobile phase with column TSK gel G3000swxl: |
| | 100 mM Potassium phosphate in combination with 200 |
| | mM KCl, pH 6.5. |
| Mode: | Isocratic |
| Detection: | UV at 215 nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

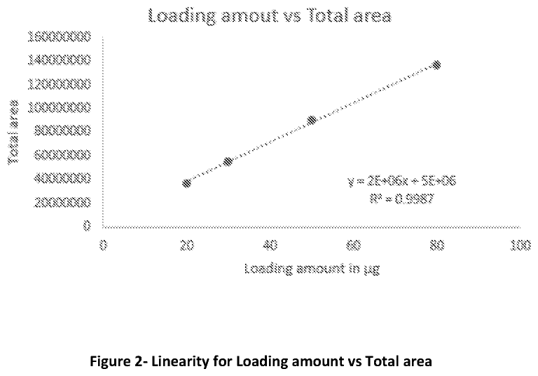

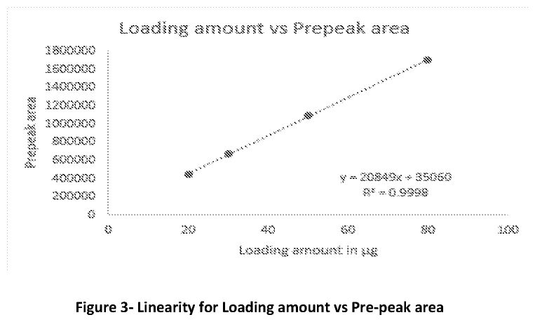

CTLA4-IgG1 fusion protein shows linear response in terms of total area (R2=0.9987) and pre-peak area (R2=0.9998) (FIGS. 2 and 3) in the range of 20 μg to 80 μg injection amount. Percentage (%) purity and total pre-peak area percentage (%) were found to be similar for all the injection amounts. Hence, it can be said that CTLA4-IgG1 fusion protein shows linearity in the load range of 20 μg to 80 μg.

TABLE 2
Results for Percentage (%) purity or main peak area percentage (%) and
total pre-peak area percentage (%) for loading amount linearity.

| | | | | Percentage (%) |
|---|---|---|---|---|
| Reference | | Area of pre- | | of Total |
| CTLA4-IgG1 | | peak HMW | | pre-peak |
| injection | Total area | impurities | Percentage | area HMW |
| amount (μg) | (μV*sec) | (μV*sec) | (%) purity | impurities |
| 20 | 36843054 | 442617 | 98.80 | 1.20 |
| 30 | 54790122 | 667094 | 98.78 | 1.23 |
| 50 | 89903649 | 1085353 | 98.79 | 1.21 |
| 80 | 136329874 | 1697915 | 98.75 | 1.24 |

### EXAMPLE 4

Solution stability for quantification of pre-peak and main peak of protein mixture containing CTLA4-IgG1 fusion protein.

During routine use of the method, it may be possible that time duration of a sample set is longer. Reference standard is generally injected in bracketing during analysis. Hence, it is necessary to check stability of diluted sample. To evaluate solution stability, sample was diluted to 1 mg/ml in mobile phase and stored at 4-8° C. in HPLC autosampler. Comparison for percentage (%) purity and percentage (%) molecular weight related impurities was done for different timepoints.

Sample (CTLA4-IgG1 fusion protein) was diluted from 25 mg/ml to 1 mg/ml in mobile phase. 30 μg sample was injected (injection volume 30 μl) from the prepared 1 mg/ml sample at time point Ohrs, 12 hrs, 24 hrs, 36 hrs and 48 hrs.

Note: 1 mg/ml sample was stored in HPLC autosampler at 4-8° C. during experiment.

### Chromatographic Conditions:

| | |
|---|---|
| HPLC system: | HPLC system equipped with a pump, an autosampler, |
| | a UV detector and a suitable data acquisition system |
| Column: | TSK gel G3000swxl |
| Mobile Phase: | Mobile phase with column TSK gel G3000swxl: |
| | 100 mM Potassium phosphate in combination with 200 |
| | mM KCl, pH 6.5 |
| Mode: | Isocratic |
| Detection: | UV at 215 nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

1 mg/ml sample when stored in HPLC autosampler at 4-8° C. and analyzed, it was observed that sample was showing similar results in terms of % purity and HMW percentage (%) up to 36 hrs. At 48 hrs, there was minor decrease in the percentage of HMWs. Hence, it can be said that diluted 1 mg/ml sample can be analyzed up to 36 hrs when stored at 4-8° C.

TABLE 3
Results for % Purity or Main peak area percentage
(%) and HMW percentage (%) for solution stability:
Results

| Time | Percentage (%) purity | HMW |
|---|---|---|
| point | or Main peak area | percentage |
| (hr) | percentage (%) | (%) |
| 0 | 99.10 | 0.90 |
| 12 | 99.17 | 0.83 |
| 24 | 99.16 | 0.84 |
| 36 | 99.19 | 0.81 |
| 48 | 99.26 | 0.74 |

Based on the result shown in Table 3, 1 mg/ml diluted sample can be analyzed up to 36 hrs when stored at 4-8° C.

### EXAMPLE 5

Size variants study for quantification of pre-peak and main peak of protein mixture containing CTLA4-IgG1 fusion protein:

SE HPLC is able to separate different size variants of CTLA4-IgG1 fusion protein. To evaluate separation of different size variants of CTLA4-IgG1 fusion protein, it needs to be generated. It was generated by doing sample treatment with DTT which can generate CTLA4-IgG1 fusion protein monomers or reduced CTLA4-IgG1 fusion protein. Different enzymatic treatment like PNGase F (generates deglycosylated form of CTLA4-IgG1 fusion protein) and IdeS (generates CTLA4 and Fc) was used. These chemically treated or enzymatically treated samples were run on SE HPLC to see the separation of different variants of CTLA4-IgG1 fusion protein.

CTLA4-IgG1 fusion protein and post-harvest samples were treated with DTT, Ides and PNGase F. These treated samples (CTLA4-IgG1 fusion protein & post-harvest samples) were diluted to 1 mg/ml in mobile phase. 30 μg sample was injected (injection volume 30 μl).

### Chromatographic Conditions:

| | |
|---|---|
| HPLC system: | HPLC system equipped with a pump, an autosampler, |
| | a UV detector and a suitable data acquisition system |
| Column: | TSK gel G3000swxl |
| Mobile Phase: | Mobile phase with column TSK gel G3000swxl: |
| | 100 mM Potassium phosphate in combination with 200 |
| | mM KCl, pH 6.5 |
| Mode: | Isocratic |
| Detection: | UV at 215 nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

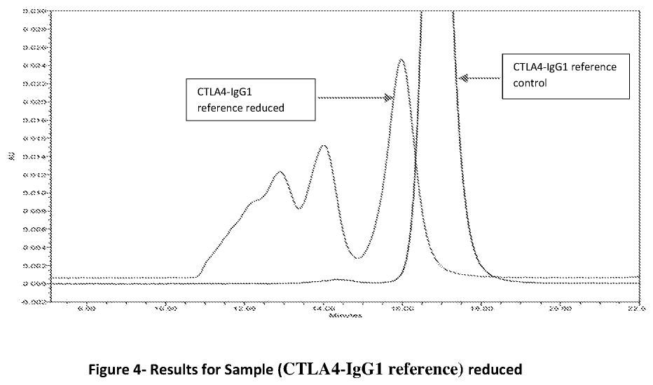

Treatment with DTT: When sample (CTLA4-IgG1 fusion protein) and post-harvest sample were treated with DTT, after reduction HMWs were increased which may be due to generation of monomers. These monomers have attached with each other by noncovalent interaction and formed the aggregates (FIGS. 4 & 5).

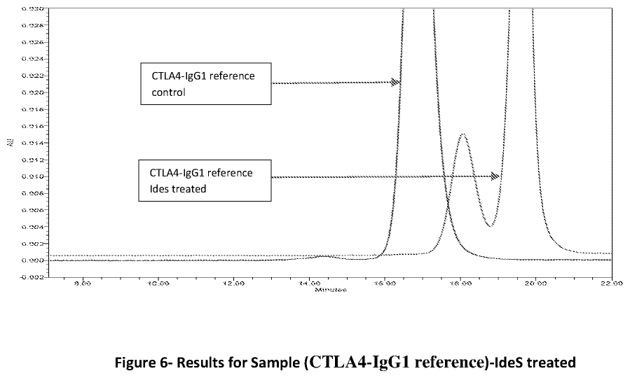

Treatment with Ides: When sample (CTLA4-IgG1 fusion protein) and post-harvest sample were treated with Ides, two fragments were generated CTLA4 and Fc. These were separated on SE HPLC as two peaks and principal peak was disappeared. (FIGS. 6 & 7)

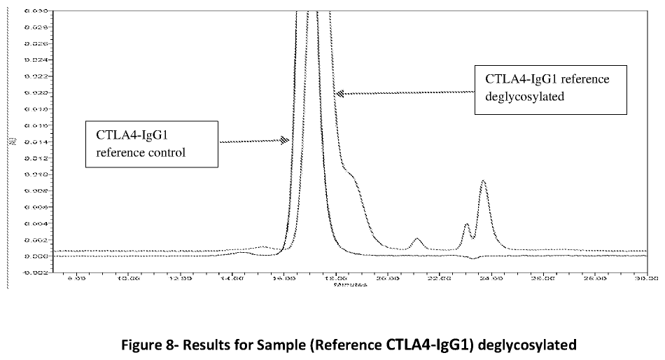

Treatment with PNGase F: Due to removal of glycans, molecular weight of CTLA4-IgG1 fusion protein was decreased, and peak has shifted towards right side (FIGS. 8 & 9).

### EXAMPLE 6

Flow rate optimization study for quantification of pre-peak and main peak of protein mixture containing CTLA4-IgG1 fusion protein:

Two different flow rates 0.5 ml/min and 0.3 ml/min were tried for SE HPLC with TSK gel G3000swxl column.

Experimental details for flow rate optimization:

| Sample preparation | Chromatographic | |
|---|---|---|
| details | conditions | Processing method |
| Reference Sample | Column: TSKgel | 0.5 ml/min flow rate- |
| (CTLA4-IgG1) was | G3000swxl Column | Integration Algorithm: Apex Track |
| diluted from 25 | Temperature: 30° C. Mode: | Start time: 10.5 min |
| mg/ml to 1 mg/ml in | Isocratic | End time: 21 min Peak width (sec): |
| mobile phase. | Run time: 60 min | 50 |
| 30 μg sample was | Detection wavelength: 215 | Detection threshold: 7.000e+01 |
| injected (injection | nm | Suitability parameter: On |
| volume 30 μl). | Mobile phase: 100 mM | 0.3 ml/min flow rate- |
| | Potassium phoshphate + | Integration Algorithm: Apex Track |
| | 200 mM KCl, pH 6.5 | Start time: 17 min |
| | Flow rate 1: 0.5 ml/min | End time: 35 min |
| | Flow rate 2: 0.3 ml/min | Peak width (sec): 50 |
| | | Detection threshold: 8.000e+01 |
| | | Suitability parameter: On |

TABLE 4
Comparative data for different flow rate:

| | Total Pre- | Total Post | | | |
|---|---|---|---|---|---|
| | peak area | peak area | | | |
| Flow rate | percentage | percentage | Percentage | Pre-peak | USP |
| (ml/min) | (%) | (%) | (%) purity | resolution | tailing |
| 0.5 | 1.29 | Not detected | 98.71 | 2.03 | 1.13 |
| 0.3 | 1.05 | Not detected | 98.94 | 2.04 | 1.10 |

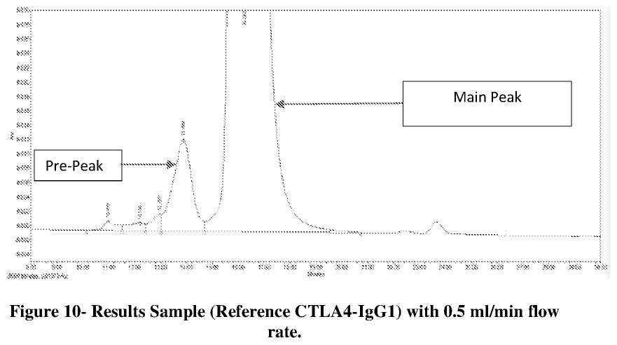

Above table 4 data shows, total pre-peak area percentage (%) was slightly lower with 0.3 ml/min flow rate as compared to 0.5 ml/min. Hence 0.5 ml/min flow rate provides improved resolution and pre-peak area. Refer FIGS. 10 & 11.

1. A method for the separation of protein mixture comprising fusion protein of interest and pre-peak impurity, the process comprises;
a) loading the protein mixture onto Size exclusion High Performance Liquid Chromatography (SE-HPLC) column;
b) separating the protein mixture with suitable mobile phase comprising combination of salts at suitable pH higher than isoelectric point (pI) of the fusion protein; wherein the mobile phase maintains flow rate more than 0.3 mL/min and less than 0.6 mL/min;
c) separating the pre-peak from fusion protein; wherein the separation provides pre-peak area not less than 1.0 and resolution more than 1.3.

2. The method according to claim 1, wherein pre-peak and fusion protein is further quantified at suitable detection absorbance selected from about 214 nm to about 280 nm.

3. The method according to claim 1, wherein the protein mixture is obtained from harvest, partially purified, substantially purified by any other purification methods.

4. The method according to claim 1, wherein the protein mixture is obtained from affinity chromatography, preferably protein A chromatography.

5. The method according to claim 1, wherein the pre-peak impurity is high molecular weight and/or aggregates.

6. The method according to claim 1, wherein the mobile phase is selected from sodium phosphate in combination with sodium sulphate, potassium phosphate in combination with potassium chloride, sodium phosphate in combination with potassium chloride, and potassium phosphate in combination with sodium sulphate in suitable concentration selected from about 50 mM to about 250 mM.

7. The method according to claim 6, wherein the mobile phase is selected from potassium phosphate & potassium chloride in suitable concentration selected from about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, and about 220 mM.

8. The method according to claim 1, wherein the mobile phase comprises salt selected from sodium sulphate, potassium chloride, in suitable concentration selected from about 50 mM to about 220 mM.

9. The method according to claim 8, wherein the salt concentration is selected from about 80 mM, about 90 mM, about 100 mM, about 110 mM and about 200 mM.

10. The method according to claim 1, wherein the suitable pH is about 5.5 to about pH 7.0, preferably about 6.5 to about 6.7.

11. The method according to claim 1, wherein the mobile phase is free of sodium chloride, arginine, acetonitrile, TFA, guanidine hydrochloride, urea and formic acid.

12. The method according to claim 1, wherein the loading of protein mixture comprises about 30 μg/μl to about 80 μg/μl.

13. The method according to claim 1, wherein the separation performed at flow rate selected from about 0.4 ml/min, about 0.5 ml/min, and about 0.6 ml/min.

14. The method according to claim 1, wherein SE-HPLC comprises size exclusion column having silica matrix, pore size selected from about 25 nm or 250 Å to about 50 nm or 500 Å and dimension selected from 150*4.6 mm, 300*7.8 mm.

15. The method according to claim 14, pore size of the SE-HPLC is 25 nm or 250 Å to 45 nm or 450 Å.

16. The method according to claim 1, wherein the size exclusion column is selected from TSKgel G3000SWXL, TSKgel G4000SWXL, TSK gel UP-SW3000, and BioSep-SEC-S2000, BioSep-SEC-S3000, BioSep-SEC-S4000.

17. The method according to claim 1, wherein the pre-peak separates within 15 minutes.

18. The method according to claim 1, wherein the peak tailing is from about 0.7 to 1.15.

19. The process as claimed in claim 1, wherein the fusion protein is selected from CTLA4-IgG1, TNFR-IgG1, VEGF-IgG1.

20. The method according to claim 1, wherein the pI of CTLA4-IgG1 is less than 6.5.
